(19) United States
(12) Patent Application Publication
(10) Pub. No.: US 2026/0083885 A1
(43) Pub. Date: Mar. 26, 2026

(54) SYSTEM AND METHOD FOR INDIVIDUALIZED PLASMA DONATION

(71) Applicant: Haemonetics Corporation, Boston, MA (US)

(72) Inventors: Michael Ragusa, Hingham, MA (US); Jan Hartmann, Brookline, MA (US); Stephen Smith, Thousand Oaks, CA (US); Seth Kasper, Orleans, MA (US)

(21) Appl. No.: 19/341,846

(22) Filed: Sep. 26, 2025

Related U.S. Application Data
(60) Provisional application No. 63/699,674, filed on Sep. 26, 2024.

(51) Int. Cl. A61M 1/02

(52) U.S. Cl. CPC A61M 1/029; A61M 2202/0014; A61M 2202/0021; A61M 2202/0415; A61M 2202/0429; A61M 2205/3303; A61M 2205/3306; A61M 2205/3327; A61M 2205/3334; A61M 2205/3368; A61M 2205/3379; A61M 2205/502; A61M 2230/005; A61M 2230/30; A61M 2230/50; A61M 2230/65

(57) ABSTRACT

A method for collecting plasma includes receiving donor information and demographic information and determining a target volume of plasma to collect based, at least in part, on the received donor information. The method also determines a plasma collection protocol based, at least in part, on the received donor information. The plasma collection protocol has a plurality of collection parameters and a plurality of process parameters. The method may then adjust at least one of the plurality of collection parameters and/or the plurality of process parameters based on the donor information and/or the demographic information. The method also performs a plasma collection procedure according to the determined plasma collection protocol and adjusted collection parameters and/or process parameters. The plasma collection procedure has a plurality of draw cycles and a plurality of return cycles.

### PRIORITY

This patent application claims priority from U.S. Provisional Application No. 63/699,674, filed Sep. 26, 2024, entitled “System and Method for Individualized Plasma Donation,” assigned attorney docket number 130670-11401, and naming Michael Ragusa, Jan Hartmann, Stephen Smith and Seth Kasper as inventors, the disclosure of which is incorporated herein, in its entirety, by reference.

### TECHNICAL FIELD

The present invention relates to systems and methods for blood apheresis, and more particularly system and methods for collecting a plasma product.

### BACKGROUND ART

Apheresis is a procedure in which individual blood components can be separated and collected from whole blood temporarily withdrawn from a subject. Typically, whole blood is withdrawn through a needle inserted into a vein of the subject's arm and into a cell separator, such as a centrifugal bowl. Once the whole blood is separated into its various components, one or more of the components (e.g., plasma) can be removed from the centrifugal bowl. The remaining components can be returned to the subject along with optional compensation fluid to make up for the volume of the removed component. The process of drawing and returning continues until the quantity of the desired component has been collected, at which point the process is stopped. A central feature of apheresis systems is that the processed but unwanted components are returned to the donor. Separated blood components may include, for example, a high density component such as red blood cells, an intermediate density component such as platelets or white blood cells, and a lower density component such as plasma.

As disclosed within U.S. Pat. No. 10,792,416 (the entirety incorporated herein by reference), the amount of plasma to be collected from a donor may be tailored to the individual donor and may be based on more than the donor's weight as traditionally done using the nomogram provided by the FDA. In that instance, the target collection volume is customized to the individual donor by taking into account the size of the donor, their body mass index, height, weight and hematocrit. This began the process of tailoring a donation to the specific characteristics of a particular donor.

### SUMMARY OF THE INVENTION

Various embodiments of the present invention continue to personalize the donation to a particular donor to not only make the donation productive in terms of product yield but also make the donation comfortable for the donor. This, in turn, results in higher donor satisfaction with the donation experience, leading to return donations (likely on a more frequent basis) which positively impacts the quality of donated product, improves the availability of plasma derived products, and generally improves healthcare and human health.

The various embodiments of the present invention are premised on the recognition that donor discomfort arises primarily from large extracorporeal volume amounts from the donor during the donation process. Various embodiments of the present invention recognize that donor comfort may be enhanced by, among other things, reducing the extracorporeal volume at any one time and by slowing the rate at which is blood drawn from the donor.

To achieve improved donor comfort, various embodiments of the present invention can do one or more of the following, alone or in combination:
[symbol] Reduce the amount of blood drawn each cycle and/or the rate at which the blood is drawn.
[symbol] Increase the number of cycles (e.g., at a lower rate and/or amount) and/or equalize the lower amount of drawn blood between the increased cycles or having cycles with differing draw amounts, in a regressive, increasing and/or stepwise progression.
[symbol] Reduce the total target plasma volume for the donation.
[symbol] Provide the donor with an instrument to control the speed of the machine (e.g., in real time) using an input device that may be tactile in nature (e.g., a squeeze ball)
[symbol] Reduce the amount of extracorporeal red blood cells drawn within a given cycle or at any point during the procedure.

The process choices and adjustments discussed above may be instituted automatically by the system by evaluation of one or more of the following data points/criteria:
[symbol] The donor's current physiological status such as age, gender, size, blood pressure, pulse, vein pressure, temperature, total blood volume, total plasma volume etc.
[symbol] The donor's personal donation history, whether the donor is new or experienced, any prior adverse events (AR), the frequency of their return, the time of day the donation is occurring, and the current wait time at the donation center.
[symbol] Demographic information that looks at the donor and the donation histories of similar donors of the same demographic in a database accessed by the system

The above data points/criteria can be evaluated together or alone and may be analyzed/synthesized using an algorithm or artificial intelligence to create a risk profile or risk factor to inform the system which process changes or remediation should be implemented for the particular donation.

In accordance with some embodiments of the present invention, a method for collecting plasma includes receiving donor information, receiving demographic information, and determining a target volume of plasma to collect and a plasma collection protocol. The target volume of plasma to collect is based, at least in part, on the received donor information. Similarly, the plasma collection protocol is based, at least in part, on the received donor information. The plasma collection protocol has a plurality of collection parameters and a plurality of process parameters. The method also adjusts at least one of the plurality of collection parameters and/or the plurality of process parameters based on the donor information and/or the demographic information. The method may then perform a plasma collection procedure according to the determined plasma collection protocol and adjusted collection parameters and/or process parameters. The plasma collection procedure has a plurality of draw cycles and a plurality of return cycles.

In some embodiments, the method may analyze the received donor information and demographic information to determine a risk assessment for the donor. The collection parameters and/or the process parameters may be adjusted according to the risk assessment. The donor information may include donor physiologic information, donor weight, donor height, donor gender, donor age, donor hematocrit, donor donation history, donor history of adverse reactions, donor blood pressure, donor pulse, donor temperature, and/or donor vein pressure. The demographic information may include demographic information of similar donors, donation histories of similar donors, and/or donation trend information.

In further embodiments, adjusting at least one of the plurality of collection parameters and/or at least one of the plurality of process parameters may include adjusting an extracorporeal volume for at least one of the plurality of draw cycles and/or reducing the collection speed for the plasma collection protocol. The extracorporeal volume may include a volume of whole blood drawn within a draw cycle and/or a volume of red blood cells withdrawn within a draw cycle. The method may also adjust the target volume of plasma to collect based on the adjusted extracorporeal volume, for example, by decreasing the target volume of plasma to collect to compensate for the adjusted extracorporeal volume. Additionally or alternatively, the method may add additional draw and return cycles to the plasma collection protocol based on the adjusted extracorporeal volume.

In some embodiments, adjusting at least one of the plurality of collection parameters and/or at least one of the plurality of process parameters may include equalizing a draw volume across each of the plurality of draw cycles and/or reducing a volume of red blood cells to be drawn during the plasma collection procedure. The method may also monitor a patient using a patient monitoring device, and further adjust at least one of the plurality of collection parameters and/or at least one of the plurality of process parameters based on the patient monitoring. The patient monitoring device may include a vein pressure monitor, a donor temperature monitor, a donor skin conductivity monitor, a camera to visually monitor the donor, and/or a donor squeeze ball.

In accordance with additional embodiments, a system for collecting plasma may include a venipuncture needle configured to draw whole blood from a donor and a blood separator configured to separate the whole blood into a plasma product and a second blood component having red blood cells. The blood separator may have a plasma output port coupled to a plasma line that sends the plasma product to a plasma product collection container. The system may also have a donor line, and anticoagulant line, and a user interface to receive input from an operator. The donor line may be fluidly coupled to the venipuncture needle and may introduce the whole blood from the donor to the blood separator. The flow through the donor line may be controlled by a first pump. The anticoagulant line may be coupled to an anticoagulant source, and may combine anticoagulant with the whole blood from the donor. The flow through the anticoagulant line may be controlled by a second pump.

A controller may be coupled to the user interface and may receive donor information and demographic information. The controller may be programmed to determine (1) a target volume of plasma to collect based, at least in part, on the received donor information, and (2) a plasma collection protocol based, at least in part, on the received donor information. The plasma collection protocol may have a plurality of collection parameters and a plurality of process parameters. The controller may also adjust at least one of the plurality of collection parameters and/or the plurality of process parameters based on the donor information and/or the demographic information. The controller may then control a plasma collection procedure according to the determined plasma collection protocol and adjusted collection parameters and/or process parameters. The plasma collection procedure may have a plurality of draw cycles and a plurality of return cycles.

In accordance with some embodiments, the controller may be further programmed to analyze the received donor information and demographic information to determine a risk assessment for the donor. The controller may adjust one or more of the plurality of collection parameters and/or the plurality of process parameters according to the risk assessment. The donor information may include donor physiologic information, donor weight, donor height, donor gender, donor age, donor hematocrit, donor donation history, donor history of adverse reactions, donor blood pressure, donor pulse, donor temperature, and/or donor vein pressure. The demographic information may include demographic information of similar donors, donation histories of similar donors, and donation trend information.

In additional embodiments, the controller may be programmed to adjust the collection parameters and/or process parameters by adjusting an extracorporeal volume for at least one of the plurality of draw cycles. The extracorporeal volume may include a volume of whole blood drawn within a draw cycle and/or a volume of red blood cells withdrawn within a draw cycle. Additionally or alternatively, the controller may adjust the target volume of plasma to collect based on the adjusted extracorporeal volume, for example, by decreasing the target volume of plasma to collect to compensate for the adjusted extracorporeal volume.

In further embodiments, the controller may add additional draw and return cycles to the plasma collection protocol based on the adjusted extracorporeal volume and/or adjust the collection parameters and/or process parameters to reduce a collection speed for the plasma collection protocol. Additionally or alternatively, the controller may equalize a draw volume across each of the plurality of draw cycles and/or reduce a volume of red blood cells to be drawn during the plasma collection procedure

The system may also include a patient monitoring device that monitors a patient during the plasma collection procedure. The controller may further adjust at least one of the plurality of collection parameters and/or at least one of the plurality of process parameters based on the patient monitoring. The patient monitoring device may include a vein pressure monitor, a donor temperature monitor, a donor skin conductivity monitor, a camera to visually monitor the donor, and/or a donor squeeze ball.

### BRIEF DESCRIPTION OF THE DRAWINGS

The foregoing features of the invention will be more readily understood by reference to the following detailed description, taken with reference to the accompanying drawings, in which:

FIG. 1 schematically shows a perspective view of a blood processing system in accordance with some embodiments of the present invention.

FIG. 2 schematically shows a top view of the blood processing system of FIG. 1, in accordance with some embodiments of the present invention.

FIG. 3 schematically shows a disposable set installed within the blood processing system of FIG. 1, in accordance with some embodiments of the present invention

FIG. 4 schematically shows the layout of a donation center having multiple blood processing systems shown in FIG. 1, in accordance with various embodiments of the present invention.

FIG. 5 is a flowchart depicting a method of collecting plasma, in accordance with some embodiments of the present invention.

FIG. 6 is a flowchart depicting a method of adjusting various collection and/or process parameters of a plasma collection procedure, in accordance with various embodiments of the present invention.

FIG. 7 schematically shows cycle volumes of a procedure operated in comfort mode and standard mode, in accordance with some embodiments of the present invention.

FIG. 8 schematically shows an example of adjusting the external blood volume using comfort mode, in accordance with some embodiments of the present invention.

FIG. 9 schematically shows an additional example of adjusting the external blood volume using comfort mode and comparison to a standard mode, in accordance with some embodiments of the present invention.

FIG. 10 schematically shows an additional exemplary comparison of a standard collection procedure and a collection procedure performed with comfort mode, in accordance with some embodiments of the present invention.

FIG. 11 schematically shows the benefits and reduction in extracorporeal volume using the comfort cycle for a number of donors, in accordance with some embodiments of the present invention.

### DETAILED DESCRIPTION OF SPECIFIC EMBODIMENTS

Illustrative embodiments of the present invention provide blood processing systems and methods for collecting a target volume of plasma (pure plasma or plasma mixed with anticoagulant). The system and method may look at a number of factors including demographic information, physiological information, donation trend information, donor information, and donation history and alter a donation procedure based on this information. For example, embodiments of the present invention may utilize the above information to alter the number of cycles in a donation, the amount of whole blood drawn and blood component (e.g., plasma) collected within a cycle, reduce the total target plasma to collect from a normal donor amount, and the maximum/peak amount of extra corporeal blood or red blood cells at a given time or within a given cycle.

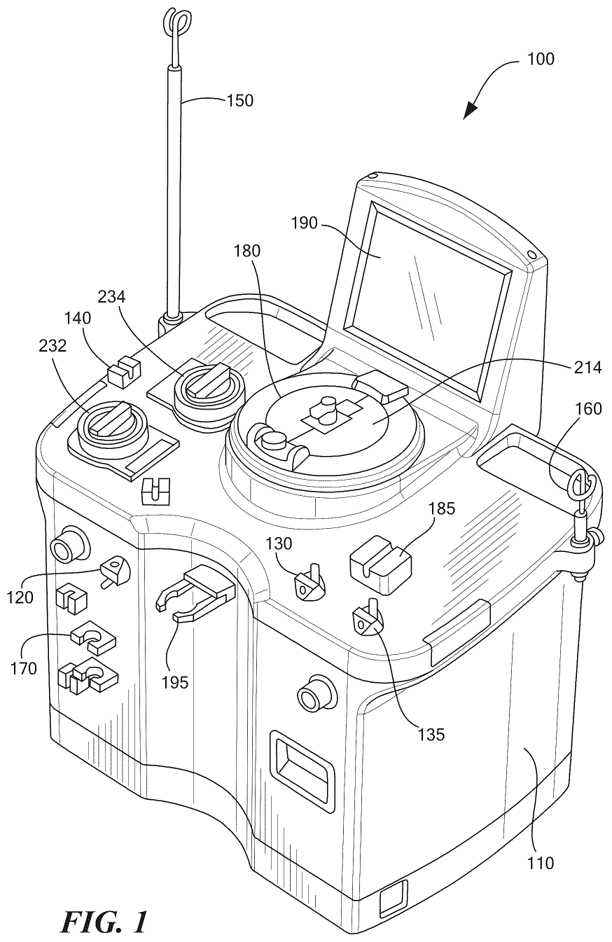

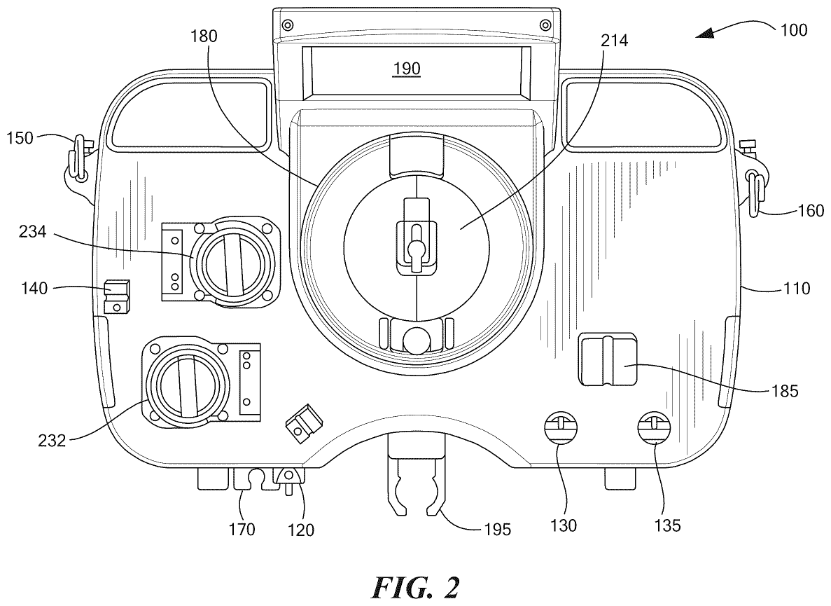

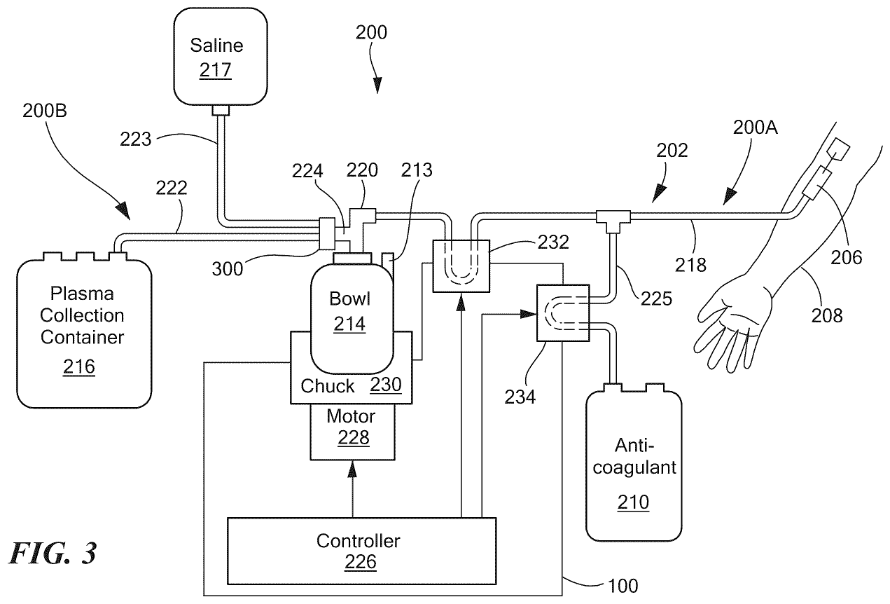

As shown in FIGS. 1 and 2, the blood processing system 100 includes a cabinet 110 that houses the main components of the system 100 (e.g., the non-disposable components). Within the cabinet 110, the system 100 may include a first/blood pump 232 that draws whole blood from a subject, and a second/anticoagulant pump 234 that pumps anticoagulant through the system 100 and into the drawn whole blood. Additionally, the system 100 may include a number of valves that may be opened and/or closed to control the fluid flow through the system 100. For example, the system 100 may include a donor valve 120 that may open and close to selectively prevent and allow fluid flow through a donor line 218 (e.g., an inlet line; FIG. 3), and a plasma valve 130 that selectively prevents and allows fluid flow through an outlet/plasma line 222 (FIG. 3). Some embodiments may also include a saline valve 135 that selectively prevents and allows saline to flow through a saline line 223.

To facilitate the connection and installation of a disposable set and to support the corresponding fluid containers, the system 100 may include an anticoagulant pole 150 on which the anticoagulant solution container 210 (FIG. 3) may be hung, and a saline pole 160 on which a saline solution container 217 (FIG. 3) may be hung (e.g., if the procedure being performed requires the use of saline). Additionally, in some applications, it may be necessary and/or desirable to filter the whole blood drawn from the subject for processing. To that end, the system 100 may include blood filter holder 170 in which the blood filter (located on the disposable set) may be placed.

As discussed in greater detail below, apheresis systems 100 in accordance with embodiments of the present invention withdraw whole blood from a subject through a venous access device 206 (FIG. 3) using the blood pump 232. As the system 100 withdraws the whole blood from the subject, the whole blood enters a blood component separation device 214, such as a Latham type centrifuge (other type of separation chambers and devices may be used, such as, without limitation, an integral blow-molded centrifuge bowl, as described in U.S. Pat. Nos. 4,983,158 and 4,943,273, which are hereby incorporated by reference). The blood component separation device 214 separates the whole blood into its constituent components (e.g., red blood cells, white blood cell, plasma, and platelets). Accordingly, to facilitate operation of the separation device 214, the system 100 may also include a well 180 in which the separation device 214 may be placed and in which the separation device 214 rotates (e.g., to generate the centrifugal forces required to separate the whole blood).

To allow the user/technician to monitor the system operation and control/set the various parameters of the procedure, the system 100 may include a user interface 190 (e.g., a touch screen device) that displays the operation parameters, any alarm messages, and buttons which the user/technician may depress to control the various parameters. Additional components of the blood processing system 100 are discussed in greater detail below (e.g., in relation to the system operation).

FIG. 3 is a schematic block diagram of the blood processing system 100 and a disposable collection set 200 (with an inlet disposable set 200A and an outlet disposable set 200B) that may be loaded onto/into the blood processing system 100, in accordance with the present invention. The collection set 200 includes a venous access device 206 (e.g., a phlebotomy needle) for withdrawing blood from a donor's arm 208, a container of anti-coagulant 210, a centrifugation bowl 214 (e.g., a blood component separation device), a saline container 217, and a final plasma collection bag 216. The blood/inlet line 218 couples the venous access device 206 to an inlet port 220 of the bowl 214, the plasma/outlet line 222 couples an outlet port 224 of the bowl 214 to the plasma collection bag 216, and a saline line 223 connects the outlet port 224 of the bowl 214 to the saline container 217. An anticoagulant line 225 connects the anti-coagulant container 210 to the inlet line 218. In addition to the components mentioned above and as shown in FIG. 3, the blood processing system 100 includes a controller 226, a motor 228, and a centrifuge chuck 230. The controller 226 is operably coupled to the two pumps 232 and 234, and to the motor 228, which, in turn, drives the chuck 230. The controller 226 may be operably coupled to and in communication with the user interface 190.

In operation, the disposable collection set 200 (e.g., the inlet disposable set 200A and the outlet disposable set 200B) may be loaded onto/into the blood processing system 100 prior to blood processing. In particular, the blood/inlet line 218 is routed through the blood/first pump 232 and the anticoagulant line 225 from the anti-coagulant container 210 is routed through the anticoagulant/second pump 234. The centrifugation bowl 214 may then be securely loaded into the chuck 230. Once the bowl 214 is secured in place, the technician may install the outlet disposable set 200B. For example the technician may connect a bowl connector 300 to the outlet 224 of the bowl 214, install the plasma container 216 into the weight senor 195, run the saline line 223 through valve 135, and run the plasma/outlet line 222 through valve 130 and the line sensor 185. Once the disposable set 200 is installed and the anticoagulant and saline containers 210/217 are connected, the system 100 is ready to begin blood processing.

Prior to connecting the donor to the blood processing device 100, it is beneficial (and perhaps necessary in some instances) to obtain/determine some information regarding the donor, namely, the donor's weight, height, age, gender, and hematocrit. Not only does this information help determine if the individual is a viable donor and the volumes of blood components that may be withdrawn/collected (e.g., per the FDA guidelines), the hematocrit may be used during processing to help collect a target volume of plasma. The technician may obtain/determine the donor's weight by weighing the donor (e.g., on a scale). To obtain/determine the donor's hematocrit, the technician may draw a blood sample from the donor and test the sample of blood. Additionally or alternatively, as discussed in greater detail below, the system may determine the hematocrit during blood processing. For example, the blood processing device 100 may include a hematocrit sensor (not shown) that determines the hematocrit of the blood flowing into the blood processing device 100 and/or the system 100 may determine the hematocrit based on a volume of red blood cells collected within the bowl 214.

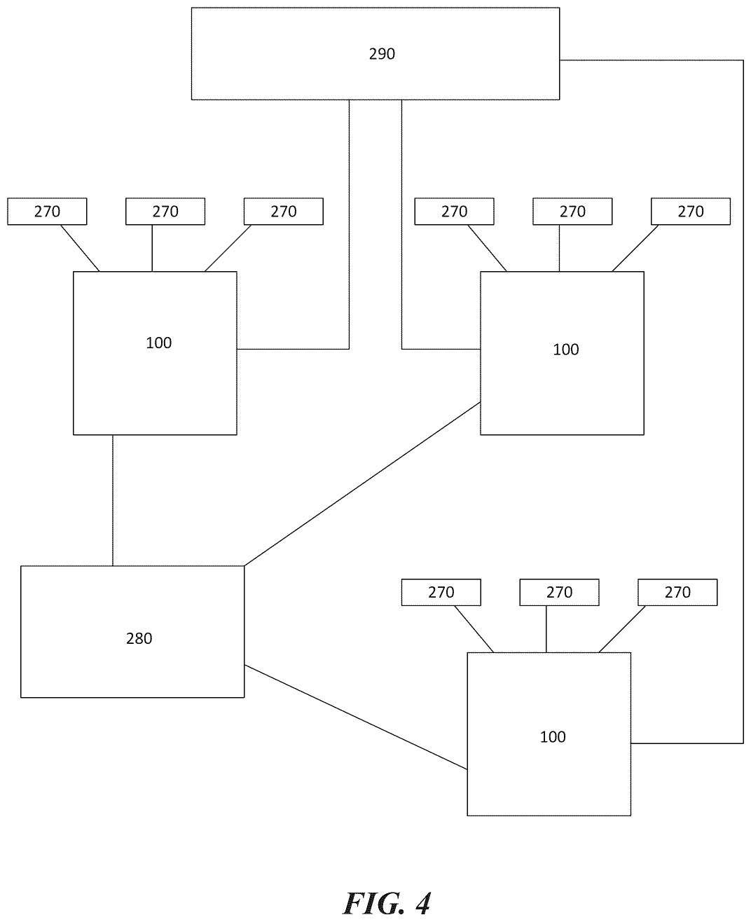

FIG. 4 schematically shows a representative donation center having multiple plasmapheresis devices/systems 100 located within the center. As discussed in greater detail below, each of the devices/systems may be associated with one or more donor monitoring devices 270 (e.g., pressure sensors, temperature sensors, cameras, squeeze balls, etc.) that may be used to monitor the comfort of the donor. As also discussed in greater detail below, each of the devices/systems 100 may be in communication with a donor management system 280 located within the donation center and/or a remote data storage system 290.

As noted above, the systems and methods described herein may use the information regarding the donor to determine the target volume of plasma product or pure plasma to collect from the donor. For example, the system/method may use the FDA approved weight based nomogram, which categorizes donors into three different weight ranges with specific maximum plasma collection targets for each range. Alternatively, the target volume of plasma may be tailored to the individual donor using the size of the donor, their body mass index, height, weight, hematocrit, total blood volume, and/or their total plasma volume.

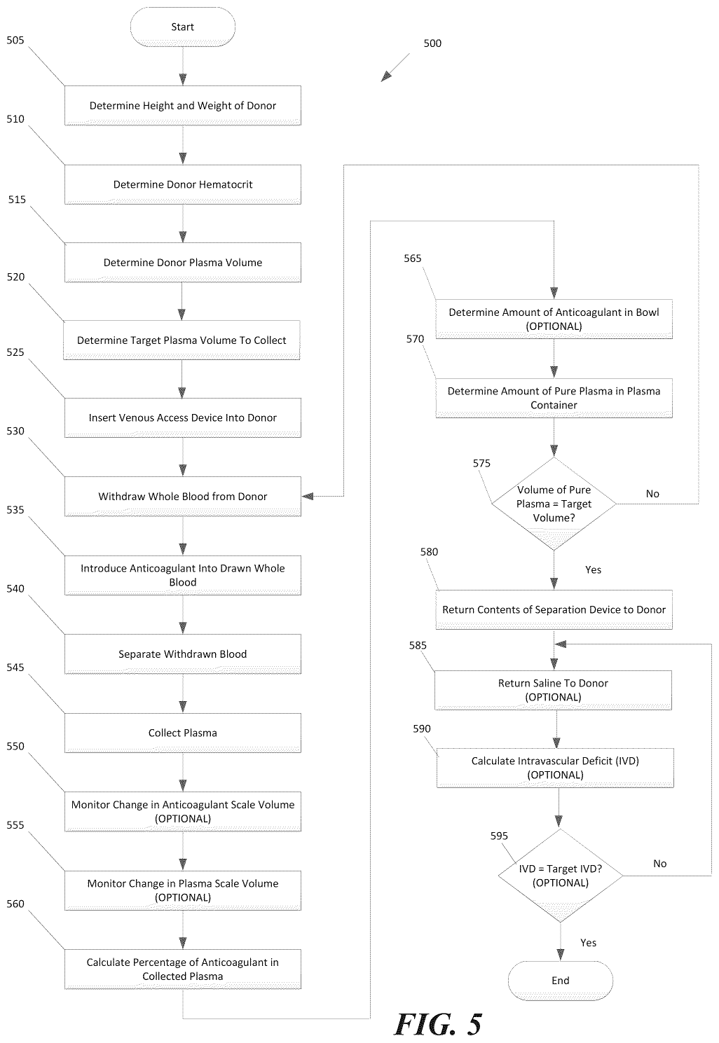

For example, as shown in FIG. 5, in embodiments that tailor the target collection volume to the individual donor, prior to connecting the donor to the blood processing device 100 and after determining the donor information, the system 100/method 500 may use the donor weight, height, hematocrit and, perhaps, gender of the donor to calculate the donor's plasma volume (e.g., the volume of plasma within the donor's blood). In such embodiments, the system 100/method 500 may determine the height, weight and hematocrit of the donor (Steps 505 and 510) and calculate the donor/subject's body mass index (“BMI”) using the donor's height and weight (e.g., BMI=weight/height²) and, in some instances, gender. The system 100 may then calculate the total blood volume within the donor/subject using the calculated BMI (e.g., see Lemmens et al., Estimating Blood Volume in Obese and Morbidly Obese Patients, Obesity Surgery, 2006:16, 773-776, the subject matter incorporated herein by reference). The total blood volume may be calculated using the following equation:

$$InBV = \frac{70}{\sqrt{} BMIp / 22}$$

In the above equation, InBV is the indexed blood volume (e.g., the donor's total blood volume), BMIp is the patient's BMI (e.g., kg/m²), 22 is the BMI value (e.g., also in kg/m²) for an ideal body weight (IBW), and 70 is the total blood volume (in mL/kg) for a donor at their ideal weight (BMI=22 kg/m²). Once the system 100 has calculated the total blood volume within the donor/subject, the system 100 (e.g., the controller 226) may then determine/calculate the volume of plasma within the donor's blood based, for example, on the donor's hematocrit (Step 515).

As mentioned above, this embodiment bases the volume of plasma to collect on the individual donor. To that end, once the system 100/method 500 has determined the donor's plasma volume, the system 100/method 500 may then calculate the target volume of plasma to collect (Step 520). For example, the system/method may multiply the total plasma volume within the patient by a target percentage of plasma to collect to obtain the target plasma volume to collect (e.g., if the total plasma volume is 2700 ml and the target percentage to collect is 28.5%, then the target plasma volume to collect is 769.5 ml). The target percentage of plasma to collect may depend on the application and/or the donor, and may be input directly into the system 100 (e.g., using the user interface 190) or may be preset from the factory. In some embodiments, the target percentage may be between 26.5 and 30%, and preferably may be 28.5%. However, in other embodiments, the target percentage may be below 26.5% or above 30% (e.g., 30.5%).

Once the lines 222/223 are in place and the technician/system has determined the necessary donor information (e.g., the donor's weight, age, gender, height and/or hematocrit), the user/technician may insert the venous access device 206 into the donor's arm 208 (Step 525). Next, the controller 226 (which controls the operation of the system 100) activates the two pumps 232, 234 and the motor 228. Operation of the two pumps 232, 234 causes whole blood to be drawn from the donor (Step 530), anticoagulant from container 210 to be introduced into the drawn whole blood (Step 535), and the now anticoagulated whole blood to be delivered to the inlet port 220 of the bowl 214.

It should be noted that the anticoagulant line 225 may also include a bacteria filter (not shown) that prevents any bacteria in the anticoagulant source 210, the anticoagulant, or the anticoagulant line 225 from entering the system 100 and/or the subject. Additionally, the anticoagulant line 225 may include an air detector 140 that detects the presence of air within the anticoagulant. The presence of air bubbles within any of the system 100 lines can be problematic for the operation the system 100 and may also be harmful to the subject if the air bubbles enter the blood stream. Therefore, the air detector may be connected to an interlock that stops the flow within the anticoagulant line 225 in the event that an air bubble is detected (e.g., by stopping the anticoagulant pump 234), thereby preventing the air bubbles from entering the subject.

As the anti-coagulated whole blood is withdrawn from the subject and contained within the blood component separation device 214, the blood component separation device 214 separates the whole blood into several blood components (Step 540). For example, the blood component separation device 214 may separate the whole blood into a first, second, third, and, perhaps, fourth blood component. More specifically, the blood component separation device 214 (and the centrifugal forces created by rotation of the separation device 214) can separate the whole blood into plasma, platelets, red blood cells (“RBC”), and, perhaps, white blood cells (“WBC”). The higher density component, i.e., RBC, is forced to the outer wall of the bowl 214 while the lower density plasma lies nearer the core. A buffy coat is formed between the plasma and the RBC. The buffy coat is made up of an inner layer of platelets, a transitional layer of platelets and WBC and an outer layer of WBC. The plasma is the component closest to the outlet port and is the first fluid component displaced from the bowl 214 via the outlet port 224 as additional anticoagulated whole blood enters the bowl 214 through the inlet port 220.

The system 100 may also include an optical sensor 213 that may be applied to a shoulder portion of the bowl 214. The optical sensor monitors each layer of the blood components as they gradually and coaxially advance toward the core from the outer wall of the bowl 214. The optical sensor 213 may be mounted in a position (e.g., within the well 180) at which it can detect the buffy coat and/or the red blood cells reaching a particular radius, and the steps of drawing the whole blood from the subject/donor and introducing the whole blood into the bowl 12 may be altered and/or terminated in response to the detection.

Additionally, in some embodiments, the optical sensor 213 may be used to determine the hematocrit of the donor during processing. For example, as the bowl 214 fills with red blood cells and the optical sensor 213 detects the layer of red blood cells, the system 100 (e.g., the controller 226) can determine the volume of red blood cells within bowl 214 based on the location of the red blood cell layer and the fixed/known bowl volume. The system 100 may then calculate the donor hematocrit based on the volume of red blood cells within the bowl and the volume of whole blood that has been processed to that point.

Once the blood component separation device 214 has separated the blood into the various components, one or more of the components can be removed from the blood component separation device 214. For instance, the plasma may be removed to and collected within a plasma container 216 (e.g., a plasma bottle) through line 222 (Step 545). As noted above, some embodiments of the system 100 may include a weight sensor 195 (FIG. 1) that measures the amount of plasma collected. The plasma collection process may continue (via a plurality of draw and return cycles) until a target volume of plasma (pure plasma or plasma mixed with anticoagulant) is collected within the plasma collection container 216. Although not shown, if the blood processing system 100 and/or the disposable set 200 include platelet, red blood cell, and/or white blood cell bags, each of the bags/containers may include similar weight sensors (e.g., load cells). It should be noted that the target volume of plasma to collect may be based on a number of donor characteristics including, not limited to, the donor's age, gender, height, weight, donation history, hematocrit, total blood volume, total plasma volume, etc. As noted above, in some embodiments, the system may utilize the donor information to determine the donor's total plasma volume and the target plasma volume may be based on a the donor's total plasma volume and target percentage of this volume to collect (as discussed within U.S. Pat. No. 10,792,416, the entirety incorporated herein by reference).

In some embodiments, the system 100 may also include a line sensor 185 (mentioned above) that can determine the type of fluid (e.g., plasma, platelets, red blood cells etc.) exiting the blood component separation device 214. In particular, the line sensor 185 consists of an LED which emits light through the blood components leaving the bowl 214 and a photo detector which receives the light after it passes through the components. The amount of light received by the photo detector is correlated to the density of the fluid passing through the line. For example, if plasma is exiting the bowl 214, the line sensor 185 will be able to detect when the plasma exiting the bowl 214 becomes cloudy with platelets (e.g., the fluid existing the bowl 214 is changing from plasma to platelets). The system 100 may then use this information to either stop the removal of blood components from the bowl 214, stop drawing whole blood from the subject, or redirect the flow by, for example, closing one valve an opening another.

As mentioned above, the target collection volume may be a volume of plasma product or pure plasma (the volume of plasma only, without any anticoagulant). For embodiments in which the target collection volume is pure plasma, it is important to note that during processing, the osmolarity of the red blood cells prevents the anticoagulant introduced into the whole blood from entering/remaining with the red blood cells (e.g., within the bowl 214). Rather, the anticoagulant mixes with the plasma component. Therefore, the anticoagulant exits the bowl 214 with the plasma and is collected within collection container 216 along with the plasma. In other words, the weight of the product measured by the weight senor 195 is the weight of the plasma, as well as any anticoagulant that is mixed with the plasma—the weight provided by the weight sensor 195 is not the weight of pure plasma.

Additionally, whole blood contains a variable amount of plasma, as determined by the donor's hematocrit. The hematocrit for typical donors can vary from 38% to 54%, which means that for 100 ml of whole blood, the volume of plasma can vary from 36 to 62 ml. Furthermore, the amount of anticoagulant added to the withdrawn whole blood is fixed (e.g., it does not depend on the hematocrit of the donor), meaning that the percentage of anticoagulant in the collected plasma may vary from 9.7% to 12.7% for donor hematocrits between 38% to 54%, respectively. Therefore, not only does the volume measured by the weight sensor 195 include the volume of anticoagulant, that volume of anticoagulant may vary from donor to donor based on the hematocrit.

To that end, some embodiments of the present invention may calculate the volume of pure plasma within the plasma collection container 216. For example, the system 100/method 500 (e.g., the controller 226) may calculate the percentage or volume of anticoagulant within the collected plasma (e.g., the plasma contained within the plasma collection container 216) (Step 560) based on the amount of anticoagulant added/metered into the whole blood and the hematocrit of the donor. The system/method can calculate the percentage or volume of anticoagulant according to the following equation, where AC is the amount of anticoagulant added to the system 100 (for example based on a ratio of AC to whole blood). As noted above, because the osmolarity of the red blood cells prevents the anticoagulant from mixing with it, essentially all of the anticoagulant exits the bowl 214 and is collected within the plasma collection container 216 along with the plasma.

$$\% A C = \frac{1}{1 + ( A C - 1 ) ( 1 - {Hct}_{D} )}$$

The amount of anticoagulant that is added to the system 100 can be determined in a number of ways. For example, the system 100 can base the amount of anticoagulant (e.g., the value of “AC” in the above equation) on the predetermined ratio of anticoagulant per unit of anticoagulated whole blood. In some embodiments, the value of “AC” may be the inverse of the predetermined ratio (e.g., “AC” would be 16 if the ratio of anticoagulant to anticoagulated whole blood was 1:16). Additionally or alternatively, the system 100 can monitor the volume of anticoagulant added to the system. In such embodiments, the system can monitor the volume of anticoagulant added to the system 100 based on the number of rotations of the anticoagulant pump (e.g., each rotation of the anticoagulant pump introduces a set volume of anticoagulant into the system 100) and/or based on the change in weight of the anticoagulant container 210 as measured by a weight sensor.

Once the system 100 has calculated the percentage or volume of anticoagulant within the plasma collection container 216, the system 100 may then use this information to calculate the volume of pure plasma within the plasma collection container 216 (Step 570). For example, the system 100 may determine the volume of anticoagulant within the container (based on the percentage or volume of anticoagulant within the container 216) and subtract this volume from the total volume of fluid within the container 216 as measured by the weight sensor 195. The system 100 may continue to monitor the volume of pure plasma collected within the container 216 and continue processing whole blood (e.g., continue performing Steps 530, 535, 540, 545, 560, 570 and, possibly, steps 550, 555 and 565, for example, in a plurality of draw and return cycles) until a volume of pure plasma is collected within the plasma collection container 216 reaches the target plasma collection volume (Step 575) (e.g., 800 mL for an adult donor weighing more than 175 pounds or other limit prescribed by the FDA or similar governing body or as calculated based on the individual plasma volume of the donor and the target percentage of plasma to collect).

As noted above, the system 100 may collect the volume of plasma in a number of cycles during which a predetermined volume of whole blood is withdrawn from the donor and separated into its components. The plasma may then be collected and the remaining contents within the bowl are returned to the donor (Step 580). If additional plasma collection cycles are to be performed (e.g., if the volume of plasma already collected does not equal the target/predetermined volume), the system 100 may once again start the blood/first pump 232 to withdraw whole blood from the subject and the system 100 may repeat the process above until the target volume of plasma is collected. The number of cycles may be determined based, for example, on the target volume of plasma to collect.

Once the system 100 has collected the target volume of plasma within the plasma collection container 216 and all of the required cycles have been completed, the system 100 can perform a final return step during which the system 100 returns the remaining components (e.g., the components remaining within the bowl 214) to the subject. For example, when all the plasma has been removed and the bowl 214 is full of RBCs (and any other blood component not collected), the controller 226 stops the draw of whole blood from the subject and reverses the direction of the blood/first pump 232 to draw the RBCs (and other components) from the bowl 214 directly back to the subject. Alternatively, if the system 100 is so equipped, the system may return the components to the subject via a dedicated return line.

It is important to note that, because the system 100/method 500 collect and do not return some of the blood components (e.g., the plasma), the volume of fluid that is returned to the donor/subject is less than the volume that was removed. This, in turn, creates an intravascular deficit that is equal to the amount of plasma collected (e.g., the volume of whole blood removed from the donor minus the volume of plasma collected/not returned). In instances in which the intravascular deficit is too large, the donor is at risk of fainting when the procedure is complete and they get up to leave the facility. To reduce the intravascular deficit (and the risk of donor injury), as noted above, some embodiments of the present invention return saline to the patient/subject (Step 585). The saline may be used as a compensation fluid to make up for the volume of the blood component (e.g., plasma) removed. To that end, during the return step (e.g., Step 580), the controller 226 (or the technician) may open the saline valve to allow saline from the saline container 217 to flow through the saline line 223 and into the bowl 214 (via outlet 224), where it can be returned to the patient/donor with or after the remaining blood components.

As mentioned above, the volume of plasma that is collected from the donor varies from donor to donor (e.g., because it is based on the donor's height, weight, hematocrit, and blood volume). Therefore, the volume of saline that is returned to the donor to reduce the intravascular deficit may similarly be dependent on the donor. To that end, when returning the contents of the separation device and the saline (Steps 580 and 585) to the donor, the system 100/method 500 may calculate the intravascular deficit (Step 590) based on the total volume of whole blood removed from the donor and the volume of blood components and saline that has been returned (or based on the volume of plasma collected and the volume of blood components and saline that have been returned). The system 100/method 500 may continue returning saline until the donor's intravascular deficit reaches a target intravascular deficit (Step 595).

The target intravascular deficit may be any intravascular deficit that reduces the donor's risk for fainting and may be the same for each donor. For example, the target intravascular deficit may be set to 0 mL or 250 mL for each donor. Alternatively, like the target plasma volume to collect, the target intravascular deficit may be different for each donor. In other words, the target intravascular deficit may be set to 0 mL for some donors and 250 mL for others. It should be noted that 0 and 250 mL are provided as examples only and other embodiments may have target intravascular deficits between 0 and 250, or greater than 250 mL. Additionally, in some instances, it may be beneficial to return more fluid to the donor than was removed/collected. In such instances, the target intravascular deficit may be set less than zero (e.g., −1 to −250 mL) so that the donor has more fluid/volume after the procedure than they did before the procedure.

The system 100/method 500 may perform some additional and optional steps to help determine the volume of pure plasma within the plasma collection container 216. For example, some embodiments may monitor the change in weight of the anticoagulant container 210 (e.g., as measured by a weight sensor/load cell on the anticoagulant container 210) (step 550). This measurement provides an indication of the volume of anticoagulant that has been added to the system 100, and may be used help determine the percentage or volume of anticoagulant within the plasma collection container 216. Additionally or alternatively, some embodiments may similarly, monitor the change in weight and/or volume of the plasma and anticoagulant collected within the plasma collection container 216 (e.g., via weight sensor 195) (step 555). This measurement may be used to calculate of the total volume of pure plasma collected within the plasma collection container 216 (e.g., to obtain the total weight from which to subtract the calculated volume of anticoagulant). Also, some embodiments may also monitor the volume of anticoagulant remaining in the bowl 214 (step 565) (e.g., anticoagulant that did not mix with the plasma and/or otherwise remained in the bowl) using the optical sensor on the bowl 214 to determine whether any anticoagulant remains within the bowl 214 and modify the calculation of the amount of pure plasma collected within the plasma collection container (e.g., either increase the calculated amount or decreased the calculated amount), based on the volume of anticoagulant remaining within the bowl 214.

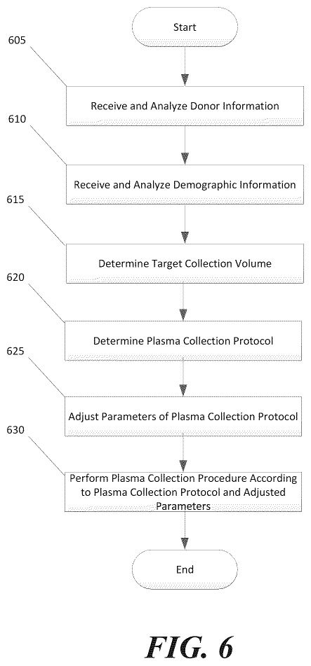

In some embodiments, the system 100 may include a comfort mode that adjusts various parameters of the plasma collection procedure (e.g., collection parameters and/or process parameters) based on donor information (e.g., donor physiologic information, donor weight, donor height, donor gender, donor age, donor hematocrit, donor donation history, donor blood pressure, donor pulse, donor temperature, and donor vein pressure) and demographic information (e.g., demographic information of similar donors, donation histories of similar donors, and donation trend information). By adjusting various parameters, embodiments utilizing the comfort mode may increase patient comfort, decrease deferral time of future donations, improve donor retention and improve donor recruitment. FIG. 6 illustrates a method of implementing the comfort mode on the system 100.

As shown in FIG. 6, the system 100/method 600 (e.g., the controller 226 of the system 100) may receive and/or analyze the donor information (step 605) and receive and/or analyze the demographic information (step 610), for example, to assess the donor's risk of an adverse reaction. The system 100/method 600 may then determine a target plasma volume to collect (Step 615), for example, based on the donor information and according to the methods for determining the target as discussed above (e.g., per the approved FDA nomogram or based on the donor's total blood volume or total plasma volume). The system 100/method 600 may then determine a plasma collection protocol for the donor based on the donor information (Step 620). The plasma collection protocol may include a number of collection parameters (e.g., the target volume to collect, draw volumes per cycle, total number of cycles, etc.) and a number of process parameters (e.g., flow rates, pressures, speed of the centrifuge bowl, etc.).

Based on the donor information and demographic information and, perhaps, the analysis of the donor information and demographic information and risk assessment, the system 100/method 600 may then adjust one or more the collection parameters and/or one or more of the process parameters (Step 625). For example, the system 100/method 600 may adjust the total extracorporeal volume or extracorporeal red blood cell volume for one or more of the draw cycles, adjust the target volume of plasma to collect, adjust the number of cycles, adjust draw rates, etc. The system 100/method 600 may then perform the plasma collection procedure according to the plasma collection protocol and the adjusted parameters (Step 630).

As mentioned, above, when utilizing the comfort mode, the system 100/method 600 may adjust and/or optimize the cycle volume (e.g., the extracorporeal volume) for a given cycle to limit the peak extracorporeal volume during the procedure. This, in turn, may increase donor comfort and reduce adverse reactions for certain donors, leading to improved donor satisfaction and donor return rates and positively impacting the overall supply of donated plasma. For example, the system 100 may minimize the extracorporeal volume for one or more cycles (e.g., some or all of the cycles) while keeping the same total number of cycles and the collection target (e.g., target plasma volume to collect) unchanged. In some instances, the system 100 may (e.g., automatically) minimize the volume drawn per cycle for low bodyweight donors as well as first time donors to reduce the risk of the donor experiencing a vasovagal reaction (VVR). Additionally, the comfort cycle/mode may equalize the volume collected and/or blood drawn in each cycle over the required number of cycles. The comfort mode may be engaged manually by the user or automatically, for example, for donors with a total blood volume of less than 3000 milliliters or based on other donor information specific to the donor or a general donor population, or demographic data. This information may be analyzed or summarized by an algorithm to provide a gradient on a scale to identify the extent that the donation procedure should be modified.

In other embodiments, if the system 100 reduces the extracorporeal volume for one or more of the cycles in a manner that may result in a reduced plasma collection amount, the system 100 may increase the number of cycles by one or more at the lower volume to ensure that the system 100 collects the same/original target collection volume. Alternatively, the system may maintain the same number of cycles at the lower extracorporeal volume and collect a reduced amount of plasma at the end of the procedure. In other embodiments, if the system identifies the donor as possibly benefiting from a more gentle collection procedure (e.g., based on the donor's physiological information, donation history, and/or risk assessment/analysis discussed in greater detail below), the system may start with a lower target collection volume and obtain this lower target collection volume by collecting a lower amount of plasma within each cycle and/or may add one or more cycle to help decrease the collection amount in each cycle.

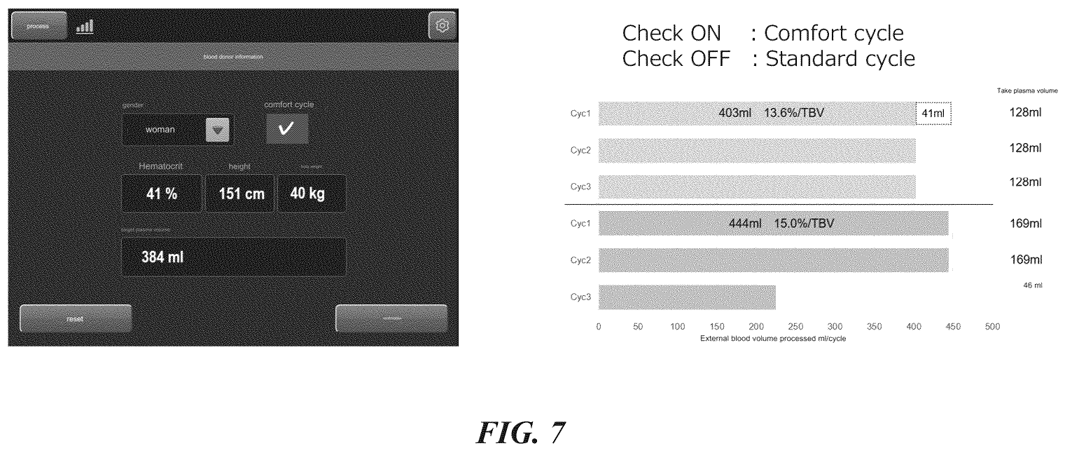

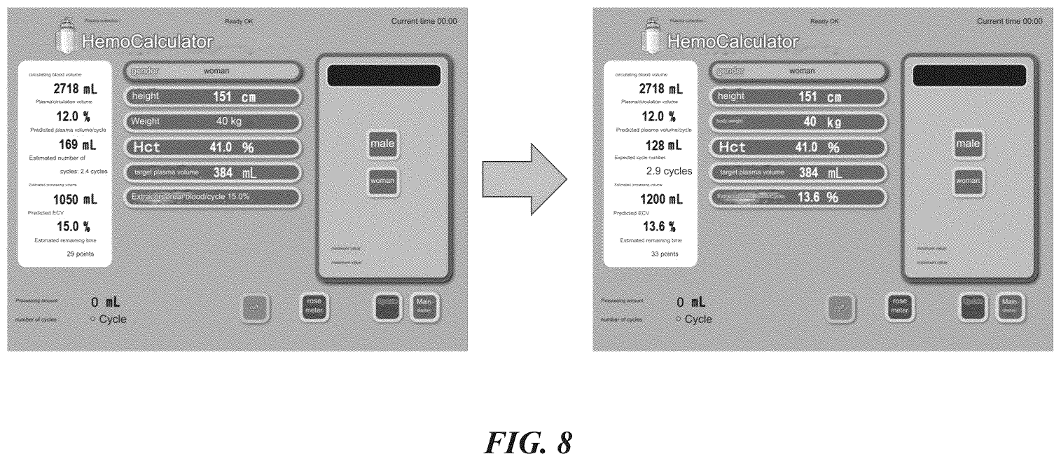

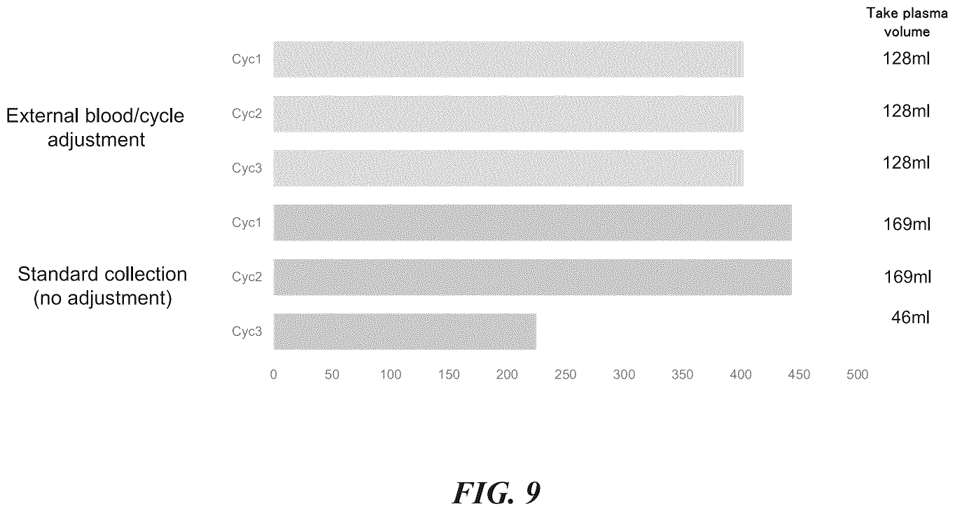

FIGS. 7 and 9 show exemplary comparisons of a standard collection cycle and a collection cycle with comfort mode activated (e.g., by selecting comfort mode on the apheresis device). In this instance, the external blood per cycle (e.g., the extracorporeal blood volume) is reduced from 15% to 13.6%, resulting in a 41 milliliter reduction per cycle. Similarly, FIG. 8 shows a reduction from 15% to 13.6% for a low body weight donor, a donor with a low circulating blood volume (low total blood volume), or a donor that is feeling unwell in order to protect against the donor having a VVR reaction. Each of the collections is performed over the course of three cycles. However, the standard collection has a 444 milliliter draw volume for the first two cycles and a much lower draw volume for the final cycle. In contrast, when the comfort mode is engaged (or the user manually reduces the extracorporeal blood volume), the draw volumes for the first two cycles are reduced (and perhaps the draw volume for the final cycle increased) to equalize the draw volumes for all three cycles to 403 milliliters. By reducing the draw volume in the first two cycles and equalizing the draw volume across all cycles, the system 100 may reduce the risk of adverse reactions/events but still collect the same volume of plasma (e.g., 384 milliliters in the examples shown in FIGS. 7-9).

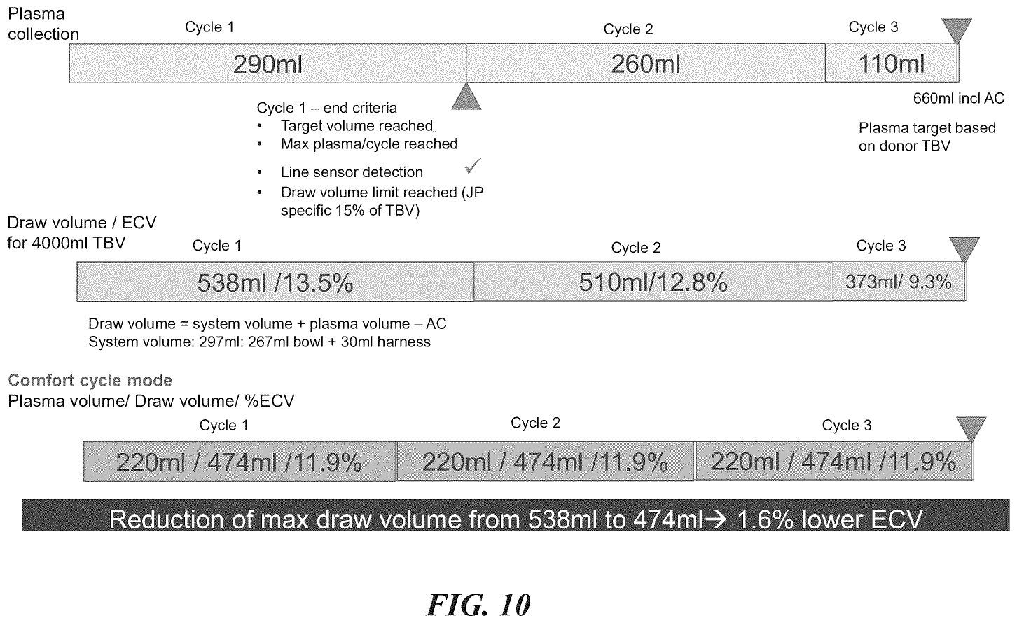

FIG. 10 also shows an additional exemplary comparison of a standard collection procedure and a collection procedure performed with comfort mode. In FIG. 10, under the standard procedure, the first cycle has a draw volume of 538 milliliters and a plasma collection volume of 290 milliliters. For the next two cycles, the draw volume drops to 510 milliliters mL and 373 milliliters and the plasma collection volume drops to 260 milliliters and 110 milliliters. In contrast, when the comfort cycle is used, the draw volume and plasma collection volume for each cycle are equalized at 474 milliliters and 220 milliliters, respectively. This results in a reduction of the maximum draw volume from 538 milliliters (for cycle 1 of the standard collection procedure) to 474 milliliters (for each of the cycles in the comfort mode collection procedure).

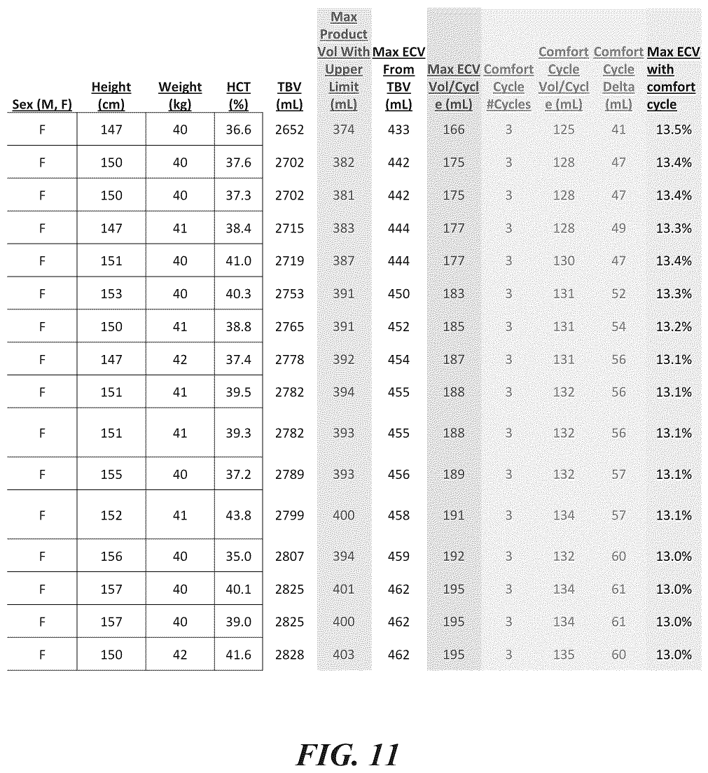

FIG. 11 is a table showing the benefits and reduction in extracorporeal volume using the comfort cycle for a number of donors with low total blood volume (TBV) and varying height, weight and hematocrit. As outlined in the table, based on the donor's total blood volume and the maximum target collection volume, the extracorporeal volume for these low total blood volume donors may be decreased when the comfort mode is engaged. The reductions may range from a 41 milliliter reduction in extracorporeal volume per cycle to a 60 milliliter reduction in extracorporeal volume per cycle.

The determination of whether or not the comfort mode should be engaged may be based on any number of factors. For example, as noted above, the comfort mode made be engaged for donors with low body weight, donors with a total blood volume below a threshold limit, and/or new donors or donors that have not donated within a determined time period. Additionally or alternatively, the comfort mode may look at a number of criteria to determine a risk factor for a given donor that is an indication of their level of risk of having an adverse event. For example, the risk factor may be based on the donor's individual characteristics (age, gender weight, total blood volume, height, hematocrit, whether the donor has had adverse reactions/events in the past, etc.), demographic data regarding which types of donors have had adverse reactions across a population of donors, and/or trends regarding which types of donors do not typically return for subsequent donations. If the risk assessment determines that a particular donor may be at risk for an adverse event under the standard protocol, the comfort mode may be used to reduce the risk.

In addition to adjusting/equalizing the draw volume and collection volume for each of the cycles of the procedure, some embodiments may also perform other functions to improve the comfort of the donor and reduce adverse events. For example, the system 100 may use the device pressure sensors to assess donor vein pressure as correlate for vasovagal tonus. By assessing vein pressure and correlating this pressure for vasovagal tonus, the system 100 may better predict adverse events and alter the procedure accordingly (e.g., by engaging comfort mode, reducing pump speeds, reducing the target plasma volume to collect, stopping the procedure, etc.). Additionally or alternatively, the system may include additional sensors that monitor/assess the donor's temperature, skin conductivity etc. (e.g., donor monitoring devices 270). This information may then be used to alter the procedure as discussed above.

In some embodiments, the system 100 may include a camera or may utilize the camera on the donor's cell phone to visually inspect/monitor the donor (e.g., additional donor monitoring devices 270). For example, by using a camera (as well as artificial intelligence to analyze the images), the system may analyze facial expressions, skin tone and pupil size to obtain information regarding the donor's comfort level and risk that an adverse event may occur. In a similar manner, the system may include a squeeze ball (e.g., donor monitoring devices 270) with an internal sensor that measures how hard and/or rapidly the donor squeezes the ball to determine the comfort level of the donor. In a manner similar to that discussed above and based on the information determined from the camera and/or squeeze ball, the system may alter the procedure by engaging comfort mode, reducing pump speeds, reducing the target plasma volume to collect, stopping the procedure, etc.

The comfort mode (e.g., the cycle limit for extracorporeal volume) may be set and/or adjusted in a number of ways. For example, the system 100 may automatically set/adjust the extracorporeal volume limits (e.g., for the comfort mode) for example, to an optimal level, based on the analysis of the donor and donation history and demographic information (e.g., based on a risk analysis similar to that discussed above). This automatic adjustment may occur particular for donors with low total blood volume. Additionally or alternatively, the technician performing the apheresis procedure may select an “ECV limit” or “cycle limit” button on the user interface to access the extracorporeal limit or cycle limit for the system. Once the technician has access the extracorporeal limit or the cycle limit they may select a preset value for the extracorporeal limit (e.g., a standard limit or limits determined by the system 100 based on the analysis of the donor information and donation history and demographic information) or the technician may manually enter a extracorporeal limit.

In addition to the comfort mode discussed above that may alter the draw volume for one or more cycles, the target collection volume, and/or add or reduce the number of cycles, some embodiments may also set or alter the extracorporeal red blood cell limit for some donors. In addition to the amount of plasma that may be collected from a donor, the FDA also has limits regarding the amount of red blood cell loss that a donor may have within a time period (e.g., if the donor has lost 200 mL of red blood cells during a plasmapheresis procedure, the donor should be deferred from another donation for up to 8 weeks). To that end, various embodiments of the present invention may review the donor information (including their prior donation records) to determine how much, if any, red blood cell loss (e.g., extracorporeal red blood cell volume) the donor has had during the prior eight weeks.

To ensure that as many red blood cells as possible are returned to the donor during plasmapheresis, the system/method may set the maximum amount of red blood cells that may be drawn by the system 100 at any point during the procedure. For example, the system 100 may include a red blood cell limit that may be adjusted to set the maximum amount of red blood cells to by drawn by the device at any point during the procedure (e.g., 15%, or less than 15% of the donor's total red blood cell volume). The amount of red blood cells that are withdrawn during a given cycle may be determined using the expected volume of whole blood to be drawn during a given cycle and the hematocrit of the donor. If the volume of red blood cells to be drawn within a given cycle exceeds the red blood cell limit, the system 100 may then alter the whole blood draw volume for one or more cycles in order to reduce the drawn red blood cell volume for the cycle to be at or below the set red blood cell limit. In a manner similar to that described above for the comfort mode, the system 100 may then increase the number of cycles in order to process the same volume of whole blood and collect the initial target collection volume or may reduce the target collection volume to compensate for the reduction in whole blood and red blood cell draw volume for any cycle that was adjusted.

The red blood cell limit may be set and/or adjusted in a number of ways. For example, the system 100 may automatically set/adjust the red blood cell based on the analysis of the donor and donation history (e.g., based on a risk analysis similar to that discussed above). Additionally or alternatively, the technician performing the apheresis procedure may select a “RBC limit” button on the user interface to access the red blood cell limit for the system. Once the technician has access the red blood cell limit, they may select a preset value for the red blood cell limit (e.g., a standard limit or limits determined by the system 100 based on the analysis of the donor information and donation history) or the technician may manually enter a red blood cell limit.

Although the red blood cell limit is particularly important for donor's who have had a recent red blood cell loss, some embodiments may also utilize the red blood cell limit for other donors to help reduce donor deferrals and adverse reactions such as donor anemia. For example, the red blood cell limit may be applied and/or adjusted if the donor is new donor or has not donated in some time, and/or at the discretion of the technician or donation center performing the plasma collection procedure.

Additionally, some embodiments may include a data transfer system that transfers various data between the plasmapheresis device and a remote system (FIG. 4) (e.g., a donor management system 280 or similar remote data storage system 290). For example, the apheresis device 100 may transfer data related to the plasmapheresis procedure (e.g., collection volumes, whether comfort mode was engaged, whether the ECV was reduced and for which cycles, whether the target collection volume was reduced, whether the red blood cell limits were used, any red blood cell loss, any alarms that may have occurred during the procedure, whether the donor had an adverse reaction, etc.) to the remote system 290. The remote system 290 may then use this information to determine parameters and changes that should be made for future donations. Additionally, the remote system 290 may also use this information to help generate any required documentation for the donation or the donation center.

It should be noted that, in addition to engaging the comfort mode or reducing the extracorporeal red blood cell volume, the risk factors discussed above may be used to modify the collection procedures for “high risk” populations. For example, based on the risk assessment, the system may change the target collection volume (e.g., to collect less from women, young donors, etc.), change the overall collection speed, change the collection speed at the beginning of the procedure, change the target volume break down by cycle (e.g., with lower volume up front/at the beginning), and/or change the number of cycles. The risk assessment (e.g., an artificial intelligence based risk assessment) could also be used to predict which donors are at risk of not returning to donate. This, in turn, may allow donation centers to have targeted interventions (provide in-center incentives and/or targeted follow up) to retain the donor and motivate them to come back. Additionally, the risk factor/assessment information, as well as the individual donor information (e.g., the physiological information of the donor, any adverse event history of the donor, donation history, etc.) may be used to develop an individual donation recommendation for the donor which, in turn, may optimize donor retention and balance safe recovery with the ability of the donor to donate. All of the information obtained and analyzed may also be added to a data repository (e.g., a cloud-based repository, remote data system 290) that may be used in ongoing/future analytics (e.g., AI analytics) and in improving future donations.

It is important to note that the donor data may be input directly into the plasmapheresis device by the user, scanned into the plasmapheresis device using a scanner (e.g., a barcode scanner at the machine) or transferred to the plasmapheresis device from a remote computer/system (e.g., a donor management system 280 or remote system 290, FIG. 4). Similarly, all of the calculations/determinations/analysis discussed above (e.g., the target plasma collection volume, the risk assessment, whether to engage comfort mode, etc.) may be performed at/by the individual plasmapheresis device or may be performed at the remote computer/system (e.g., the donor management system 280 or remote system 290, FIG. 4) and then sent (or otherwise transferred) to the individual plasmapheresis device. In other embodiments, the calculations/determinations/analysis may be performed on an application run on the donor's phone/tablet or the system operator's phone/tablet.

By incorporating the features discussed above and by reducing the amount of extracorporeal blood for one or more cycles and/or reducing the amount of extracorporeal red blood cells during the plasmapheresis procedure, various embodiments of the present invention improve the comfort of the donor, improve the donor experience, and reduce the risk of red blood cell loss during the procedure. This, in turn, helps to recruit and retain donors (e.g., so that the donors will return for future donations) and decreases the likelihood that the donor will need to be deferred from future donations based on red blood cell loss. Additionally, by basing the target collection volume on the individual donor characteristics such as their total blood volume or total plasma volume and collecting a target volume of pure plasma (plasma only without anticoagulant), various embodiments are able to maximize the volume of plasma collected across a population of donors.

In some embodiments, the system 100 may analyze the donor data and/or the demographic data discussed above to determine if a given procedure will be likely to result in a plasma product that is suitable for fresh frozen plasma (FFP) transfusion. For example, if, based on the analysis and the expected procedure (e.g., the selected procedure and adjusted collection/process parameters), the system 100 determines that the procedure will result in a target collection amount that is above a threshold (e.g., 690 milliliters for a transfusable dose), that the number of cycles (e.g., the equalized cycles) fall within a predetermined range of cycles, and the likelihood that the line sensor 185 will not trip/be triggered (e.g., that that the specific procedure provides sufficient buffer from the possibility of the line sensor 185 detecting the presence of platelets, white blood cells, and/or buffy coat leaving the bowl 214), the system 100 may flag the upcoming procedure as suitable for FFP (e.g., that the procedure is capable of being suitable and/or optimized for use in a subsequent FFP transfusion).

By providing a buffer from the line sensor 185 trips (e.g., an acceptable likelihood that the line sensor 180 will not trip and/or detect platelets, white blood cells and/or buffy coat leaving the bowl 214), the system 100 helps ensure that the quality of the collected plasma product is above a certain level/threshold (e.g., that the collected plasma has a low level of platelets or white blood cells (e.g., buffy coat) such that it is suitable for an FFP transfusion). If the calculated/set buffer is within a sufficient (and configurable) range for each cycle (e.g., greater than 5-10 milliliters before the platelets, white blood cells and/or buffy coat are expected to exit the bowl 214) then the system 100 may determine that the given procedure/cycle has less likelihood to trip on the line sensor 185, and instead each of the cycles would end on calculated volume (rather than the line sensor 185 detecting the presence of platelets or white blood cells (e.g., buffy coat)) and the system 100 taking action based on the presence of platelets or white blood cells (e.g., buffy coat) (as discussed above). When each of the cycles ends based on volume and not the tripping of the line sensor (e.g., the detection of platelets/white blood cells/buffy coat), the system 100 may consider the white blood cell content of the end product to be sufficiently low and that it may be utilized for FFP.

In addition to adjusting various process and collection parameters of the procedure, some embodiments may utilize the donor data and/or the demographic data to determine what type of disposable set may be used for the donor's procedure. For example, if the analysis of the donor data and/or the demographic data suggests that the donor is at risk of an adverse reaction, the quality of the plasma will not be suitable for a FFP transfusion (discussed above), the volume of plasma collected will be below a threshold (e.g., insufficient for an FFP transfusion or below a target collection volume) or there is a risk that the donation procedure may be unsuccessful (e.g., the procedure has to be stopped early, etc.), the system 100 can recommend that an unbundled disposable set (e.g., a lower cost set) be used. The unbundled set may be an unconnected set that is assembled/connected on site and may include components such at the needle, a whole blood sampling device, the donor line 218, the bowl 214, and a plasma collection container 216 (e.g., a bottle or bag). Conversely, if the analysis of the donor data and/or demographic data suggests that there is a strong likelihood of a successful donation (e.g., the quality and quantity of plasma collected will be suitable for an FFP transfusion, low risk of donor reactions, low risk of the procedure needing to be stopped), the system 100 may suggest that a bundled disposable set (e.g., a more expensive set) should be used. The bundled set may be an entire pre-connected set (e.g., that does not need to be assembled on site) and may include the needle, whole blood sampling device, the donor line 218, bowl 214, air bag, and a plasma collection container 216 (e.g., a bottle or bag).

By pre-selecting donors which are less likely to have line sensor trips during the collection procedure (e.g., such that the collected plasma is suitable for FFP procedures), the costs for end product screening may be reduced. Additionally, as noted above, the costs may be further managed by utilizing the unbundled collection disposables (lower cost option) when line sensor trips are likely to occur (as opposed to ending the cycles/procedure based on collected volume) or the collection volume is insufficient for an FFP threshold. The system 100 may suggest the higher cost option (bundled kits) when the system 100 determines that a good margin (e.g., below a threshold level) against line sensor trips exists and therefore the investment in the given collection procedure is further validated. In some instances, the system 100 may include a setting that suggests a particular disposable kit based on the output of the buffer for the line sensor 185. Furthermore, particular geographic regions or donation centers may select the appropriate disposable set configuration the system 100 should suggest based on the pre-calculated buffer decision. The decision points may be a sufficient buffer such that the line sensor 185 does not trip (e.g., it does not detect the presence of white blood cells, platelets or buffy coat leaving the bowl 214), total plasma product available, thresholds for a local unit size of FFP, etc. In some instances, the system 100 may suggest that an unbundled disposable set be used for larger donors to help ensure that a maximum amount of plasma is collected.

It is also important to note that, although the various embodiments discussed above are in relation to a blood processing system that collects plasma, the features discussed herein may be applied to any type of blood processing system. For example, the features described herein may be implemented on blood processing systems that collect and/or process red blood cells, platelets and/or white blood cells.

The embodiments of the invention described above are intended to be merely exemplary; numerous variations and modifications will be apparent to those skilled in the art. All such variations and modifications are intended to be within the scope of the present invention as defined in any appended claims.

What is claimed is:

1. A method for collecting plasma comprising:
receiving donor information;
receiving demographic information;
determining a target volume of plasma to collect based, at least in part, on the received donor information;
determining a plasma collection protocol based, at least in part, on the received donor information, the plasma collection protocol having a plurality of collection parameters and a plurality of process parameters;
adjusting at least one of the plurality of collection parameters and/or the plurality of process parameters based on the donor information and/or the demographic information; and
performing a plasma collection procedure according to the determined plasma collection protocol and adjusted collection parameters and/or process parameters, the plasma collection procedure having a plurality of draw cycles and a plurality of return cycles.

2. The method according to claim 1, further comprising:
analyzing the received donor information and demographic information to determine a risk assessment for the donor, wherein the at least one of the plurality of collection parameters and/or the plurality of process parameters is adjusted according to the risk assessment.

3. The method according to claim 1, wherein the donor information includes at least one selected from the group consisting of donor physiologic information, donor weight, donor height, donor gender, donor age, donor hematocrit, donor donation history, donor history of adverse reaction, donor blood pressure, donor pulse, donor temperature, and donor vein pressure.

4. The method according to claim 1, wherein the demographic information includes at least one selected from the group consisting of demographic information of similar donors, donation histories of similar donors, and donation trend information.

5. The method according to claim 1, wherein adjusting at least one of the plurality of collection parameters and/or at least one of the plurality of process parameters includes adjusting an extracorporeal volume for at least one of the plurality of draw cycles.

6. The method according to claim 5, wherein the extracorporeal volume includes a volume of whole blood drawn within a draw cycle and/or a volume of red blood cells withdrawn within a draw cycle.

7. The method according to claim 5, further comprising:
adjusting the target volume of plasma to collect based on the adjusted extracorporeal volume.

8. The method according to claim 7, wherein adjusting the target volume of plasma to collect based on the adjusted extracorporeal volume includes decreasing the target volume of plasma to collect.

9. The method according to claim 8, wherein the target volume of plasma to collect is decreased to compensate for the adjusted extracorporeal volume.

10. The method according to claim 5, further comprising:
adding additional draw and return cycles to the plasma collection protocol based on the adjusted extracorporeal volume.

11. The method according to claim 1, wherein adjusting at least one of the plurality of collection parameters and/or at least one of the plurality of process parameters includes reducing a collection speed for the plasma collection protocol.

12. The method of claim 1, wherein adjusting at least one of the plurality of collection parameters and/or at least one of the plurality of process parameters includes equalizing a draw volume across each of the plurality of draw cycles.

13. The method according to claim 1, wherein adjusting at least one of the plurality of collection parameters and/or at least one of the plurality of process parameters includes reducing a volume of red blood cells to be drawn during the plasma collection procedure.

14. The method according to claim 1, further comprising:
monitoring a patient using a patient monitoring device; and
further adjusting at least one of the plurality of collection parameters and/or at least one of the plurality of process parameters based on the patient monitoring.

15. The method according to claim 14, wherein the patient monitoring device includes at least one selected from the group consisting of a vein pressure monitor, a donor temperature monitor, a donor skin conductivity monitor, a camera to visually monitor the donor, and a donor squeeze ball.

16. A system for collecting plasma comprising:
a venipuncture needle configured to draw whole blood from a donor;
a blood separator configured to separate the whole blood into a plasma product and a second blood component comprising red blood cells, the blood separator having a plasma output port coupled to a plasma line configured to send the plasma product to a plasma product collection container;
a donor line fluidly coupled to the venipuncture needle configured to introduce the whole blood from the donor to the blood separator, flow through the donor line being controlled by a first pump;
an anticoagulant line coupled to an anticoagulant source, the anticoagulant line configured to combine anticoagulant with the whole blood from the donor, flow through the anticoagulant line being controlled by a second pump;
a user interface configured to receive input from an operator; and
a controller coupled to the user interface and configured to receive donor information and demographic information, the controller programmed to (1) determine a target volume of plasma to collect based, at least in part, on the received donor information, (2) determine a plasma collection protocol based, at least in part, on the received donor information, the plasma collection protocol having a plurality of collection parameters and a plurality of process parameters, (3) adjust at least one of the plurality of collection parameters and/or the plurality of process parameters based on the donor information and/or the demographic information, and (4) control a plasma collection procedure according to the determined plasma collection protocol and adjusted collection parameters and/or process parameters, the plasma collection procedure having a plurality of draw cycles and a plurality of return cycles.

17. The system according to claim 16, wherein the controller is further programmed to analyze the received donor information and demographic information to determine a risk assessment for the donor, wherein the controller adjusts the at least one of the plurality of collection parameters and/or the plurality of process parameters according to the risk assessment.

18. The system according to claim 16, wherein the donor information includes at least one selected from the group consisting of donor physiologic information, donor weight, donor height, donor gender, donor age, donor hematocrit, donor donation history, donor history of adverse reaction, donor blood pressure, donor pulse, donor temperature, and donor vein pressure.

19. The system according to claim 16, wherein the demographic information includes at least one selected from the group consisting of demographic information of similar donors, donation histories of similar donors, and donation trend information.

20. The system according to claim 16, wherein the controller is programmed to adjust at least one of the plurality of collection parameters and/or at least one of the plurality of process parameters by adjusting an extracorporeal volume for at least one of the plurality of draw cycles.

21. The system according to claim 20, wherein the extracorporeal volume includes a volume of whole blood drawn within a draw cycle and/or a volume of red blood cells withdrawn within a draw cycle.

22. The system according to claim 20, wherein the controller is further programmed to adjust the target volume of plasma to collect based on the adjusted extracorporeal volume.

23. The system according to claim 22, wherein the controller adjusts the target volume of plasma to collect based on the adjusted extracorporeal volume by decreasing the target volume of plasma to collect.

24. The system according to claim 23, wherein the controller decreases the target volume of plasma to collect to compensate for the adjusted extracorporeal volume.

25. The system according to claim 20, wherein the controller is further programmed to add additional draw and return cycles to the plasma collection protocol based on the adjusted extracorporeal volume.

26. The system according to claim 16, wherein the controller is further programmed to adjust at least one of the plurality of collection parameters and/or at least one of the plurality of process parameters to reduce a collection speed for the plasma collection protocol.

27. The system of claim 16, wherein the controller is further programmed to equalize a draw volume across each of the plurality of draw cycles.

28. The system according to claim 16, wherein the controller is further programmed to reduce a volume of red blood cells to be drawn during the plasma collection procedure.

29. The system according to claim 16, further comprising:
a patient monitoring device configured to monitor a patient during the plasma collection procedure, the controller programmed to further adjust at least one of the plurality of collection parameters and/or at least one of the plurality of process parameters based on the patient monitoring.

30. The system according to claim 29, wherein the patient monitoring device includes at least one selected from the group consisting of a vein pressure monitor, a donor temperature monitor, a donor skin conductivity monitor, a camera to visually monitor the donor, and a donor squeeze ball.
